(19) United States
(12) Patent Application Publication
(10) Pub. No.: US 2006/0275873 A1
(43) Pub. Date: Dec. 7, 2006

(54) Autoinducer compound to improve the productivity of natamycin streptomyces strains

(76) Inventors: Eliseo Recio Perez, Navatejera (ES); Angel Jose Colina Delgado, Sariegos (ES); Jesus Manuel Fernandez Aparicio, Villaobispo De Las Regueras (ES); Juan Francisco Martin Martin, Leon (ES)

Correspondence Address: NIXON & VANDERHYE, PC, 901 NORTH GLEBE ROAD, 11TH FLOOR, ARLINGTON, VA, 22203, US

(21) Appl. No.: 10/557,230

(22) PCT Filed: May 13, 2004
(86) PCT No.: PCT/EP04/05187

§ 371 Date: Nov. 16, 2005

(30) Foreign Application Priority Data
May 22, 2003 (EP) 03076559.8

(51) Int. Cl. C12P 19/62; C12N 1/21; C07C 215/02

(52) U.S. Cl. 435/76; 435/252.35; 564/506

(57) ABSTRACT

The invention provides a compound of the formula (I). Furthermore, the invention provides a process for the production of a compound of formula (I). The present invention also provides a process for the production of natamycin by a Streptomyces strain comprising adding a composition comprising an auto inducer to the fermentation medium. The present invention furthermore provides the use of a compound of formula (I) in the manufacture of a product by fermentation of a Streptomyces strain. The present invention finally discloses a Streptomyces strain that is defective in natamycin production and that is capable of producing a compound of formula (I) wherein both R₁ and R₂ are hydrogen and a Streptomyces strain that is defective in production of a compound of formula (I) wherein both R₁ and R₂ are hydrogen and that is capable of producing natamycin in the presence of said compound.

### FIELD OF THE INVENTION

The present invention relates to the fermentative production of compounds such as secondary metabolites, proteins or peptides. More specifically, the present invention relates to compounds that improve the productivity of natamycin-producing Streptomyces strains.

### BACKGROUND OF THE INVENTION

Actinomycetes, a family of filamentous bacteria, produce a wide variety of secondary metabolites including the polyene macrolides. Polyene macrolides are antifungal compounds synthesized by more than one hundred different species of Actinomycetes. From a biosynthetic point of view, these compounds are a subclass of the widely distributed polyketides. Well-known examples of the polyene macrolides are amphotericin B, natamycin (also referred to as pimaricin) and nystatin.

For obtaining these products, the bacteria are generally cultivated in liquid media (submerged cultures) leading to excretion of the products into the liquid, from which they can be isolated. Formation of product can take place during the initial fast growth of the organism and/or during a second period in which the culture is maintained in a slow-growing or non-growing state. During such a process, the amount of product which is formed per unit of time (the productivity) is generally a function of a number of factors: the intrinsic metabolic activity of the organism; the physiological conditions prevailing in the culture (e.g. pH, temperature, medium composition); and the amount of organisms which are present in the equipment used for the process. Generally, during optimization of a fermentation process, the focus is on obtaining the highest possible productivity. One solution to this problem is obtaining a concentration of bacteria that is as high as possible. However, one particular characteristic of bacteria that belong to the family of Actinomycetes makes it difficult to achieve this goal. Actinomycetes, when grown in submerged culture, have a filamentous morphology, which generally leads to highly viscous culture fluids. Another solution to the problem of obtaining a high productivity may be the development of new strategies to improve the productivity of the Actinomycetes. This would mean that the same process could be operated at a higher production rate and/or it would be possible to achieve a higher concentration of product. Both changes in the process will result in higher process productivity. There exists therefore a need for new strategies for improvement of the productivity in fermentation processes comprising Actinomycetes.

### SUMMARY OF THE INVENTION

The present invention provides a compound of the formula (I): 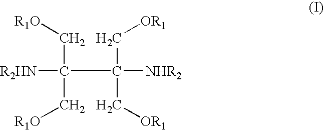
wherein:
each R₁ is hydrogen, optionally substituted alkyl, substituted silyl or —C(O)(R₃) wherein R₃ is hydrogen, optionally substituted alkyl, or optionally substituted aryl; and
each R₂ is hydrogen, optionally substituted alkyl, substituted silyl or —C(O)(R₃) wherein R₃ is hydrogen, optionally substituted alkyl, optionally substituted aryl or OR₄ wherein R₄ is optionally substituted alkyl, or optionally substituted aryl; or a salt thereof.

Each of R₃ and R₄ are preferably methyl, ethyl, n-propyl, or isopropyl.

Furthermore, the present invention provides a process for the production of a compound of formula (II): 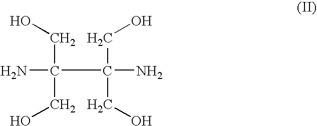
comprising fermenting a Streptomyces strain capable of producing said compound of formula (II). The method may also comprise isolating said compound of formula (II) from the mixture obtained. The isolated compound of formula (II) may then be purified.

Additionally, the present invention provides processes for the production of a compound of formula (I), wherein R₁ and R₂ are not both hydrogen, comprising reacting a compound of formula (II) with acetylating agents.

The present invention also provides a fermentation process for the production of natamycin by a Streptomyces strain comprising the addition of a composition comprising an auto inducer to the fermentation medium.

The present invention further provides a fermentation process for the production of natamycin by a Streptomyces strain comprising increasing the concentration of an auto inducer in the fermentation medium by increasing the natural production of said auto inducer by said Streptomyces strain.

The present invention furthermore provides the use of a compound of formula (I) in the manufacture of a product by fermentation of a Streptomyces strain.

The present invention also provides a Streptomyces strain that is defective in natamycin production and that is capable of producing a compound of formula (II). The invention further provides a Streptomyces strain that is defective in production of a compound of formula (II) and that is capable of producing natamycin in the presence of said compound of formula (II).

### DETAILED DESCRIPTION OF THE INVENTION

The terms and abbreviations given below are used throughout this disclosure and are defined as follows.

The term ‘A factor’ refers to 2-(6′-methylheptanoyl)-3R-hydroxymethyl-4-butanolide, which is the natamycin-inducing factor from Streptomyces griseus.

The term ‘FMOC’ refers to 9-fluorenylmethyloxycarbonyl.

The term ‘IP factor’ refers to 2,3-diamino-2,3-bis(hydroxymethyl)-1,4-butanediol.

The term ‘npi’ refers to non-producer mutants that are impaired in natamycin biosynthesis.

The term ‘MEA’ refers to malt extract media.

The term ‘NBG’ refers to peptone-beef extract media.

The term ‘NTG’ refers to mutagenesis using N-methyl-N′-nitrosoguanidine.

The term ‘TSB’ refers to tryptone soy broth extract.

The term ‘YED’ refers to yeast extract-dextrose media.

The term ‘YEME’ refers to yeast extract-malt extract media.

Natamycin represents a prototype molecule of glycosylated polyenes that is important for antifungal therapy. Natamycin also displays antiviral activity, stimulates the immune response and acts in synergy with other antifungal drugs or anti-tumor compounds. Natamycin is produced by Streptomyces strains such as Streptomyces natalensis and Streptomyces gilvosporeus and is widely utilized in the food industry to prevent mold contamination of cheese and other non-sterile foods (i.e. cured meats).

Surprisingly we have found, isolated and characterized a group of novel compounds that, when added to a natamycin-producing organism, may increase natamycin productivity by 20 to 65%. One such compound, 2,3-diamino-2,3-bis(hydroxy-methyl)-1,4-butanediol (hereafter referred to as IP factor), is symmetrical and has an entirely novel chemical structure.

Diffusible low molecular weight chemical substances called auto inducers control secondary metabolism and cell differentiation in Actinomycetes. There are different types of auto inducing factors, which belong to at least five chemical classes. These are as follows:
1) the butyrolactone class, which includes the A factor of Streptomyces griseus, the virginia butanolide factors of Streptomyces virginiae and similar compounds isolated from Streptomyces coelicolor, Streptomyces viridochromogenes, Streptomyces bikiniensis, Streptomyces cyaneofuscatus, Vibrio fischeri and other Actinomycetes. The structure of these compounds are described in Horinouchi et al. (Mol. Microbiol. 12, 859-864, 1994);
2) the homoserine lactones of Gram-negative bacteria;
3) the nucleotide-like B factor (3′-(1-butylphosphoryl)adenosine) of rifamycin-producing Amycalotopsis (Nocardia) mediterranei;
4) furanosyl borate diester, which is a quorum sensing inducer in Gram-negative bacteria; and
5) the modified peptide pheromones of Gram-negative and Gram-positive bacteria.

Surprisingly, there is no structural relationship between compounds disclosed herein and any of the abovementioned classes of auto inducers.

In a first aspect of the invention is described a compound of the formula (I): 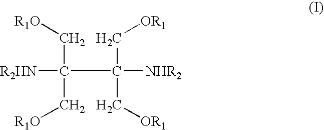
wherein:
each R₁ is hydrogen, optionally substituted alkyl, substituted silyl or —C(O)(R₃) wherein R₃ is hydrogen, optionally substituted alkyl, or optionally substituted aryl; and
each R₂ is hydrogen, optionally substituted alkyl, substituted silyl or —C(O)(R₃) wherein R₃ is hydrogen, optionally substituted alkyl, optionally substituted aryl or OR₄ wherein R₄ is optionally substituted alkyl, or optionally substituted aryl; or salts thereof. Preferably said salts are salts from inorganic acids such as carbon dioxide, phosphorous acid, hydrobromic acid, hydrochloric acid, nitric acid, perchloric acid, sulfuric acid and the like or from organic acids such as acetic acid, formic acid, oxalic acid and the like.

Each of R₃ and R₄ are preferably methyl, ethyl, n-propyl, or isopropyl.

Optionally substituted alkyl groups are straight chain alkyl groups from C₁₋₂₀, preferably C₁₋₁₂, more preferably C₁₋₈, most preferably C₁₋₅ that may or may not be substituted at one or more positions in the chain with other groups that may be alkyl, aryl, amino, hydroxyl and/or sulfur groups. Likewise, optionally substituted aryl groups are aromatic groups such as benzene, pyridine, thiazoles and the like that may or may not be substituted at one or more positions in the ring with other groups that may be alkyl, aryl, amino, hydroxyl and/or sulfur groups. Silyl groups are generally substituted with simple alkyl chains such as methyl, ethyl, isopropyl, tert-butyl or with aryl groups such as phenyl.

In one embodiment, both R₁ and R₂ are hydrogen and the compound is referred to as IP factor.

In another embodiment, R₁ is —C(O)(R₃) wherein R₃ is hydrogen, optionally substituted alkyl, or optionally substituted aryl and R₂ is hydrogen. In this embodiment R₃ is preferably methyl, ethyl, n-propyl, isopropyl. Such acetylated derivatives of IP factor are valuable for NMR structure elucidating studies and can be used as intermediates in synthetic methods that produce IP factor. Said compounds, which are esters, can be obtained by reacting IP factor with a variety of acetylating agents that are known to the person skilled in the art (for instance, March “Advanced Organic Chemistry”, John Wiley & Sons, Inc, 1985). In some cases, these reactions require a protection-deprotection sequence for the amino groups. Said acetylating agents may be carboxylic acids, which may require the presence of catalysts such as strong organic acids; acid halides such as acetyl chloride; or acid anhydrides such as acetic anhydride. Preferably said compounds are of the formula (I) wherein R₁ is —C(O)(CH₃) and R₂ is hydrogen (tetra-acetyl IP factor, IPa) or of the formula (I) wherein R₁ is —C(O)(CH₂CH₃) and R₂ is hydrogen.

In yet another embodiment, R₁ is hydrogen and R₂ is —C(O)(R₃) wherein R₃ is hydrogen, optionally substituted alkyl or optionally substituted aryl or OR₄ with R₄ is optionally substituted alkyl, or optionally substituted aryl or salts thereof. Each of R₃ and R₄ are preferably methyl, ethyl, n-propyl, isopropyl. Such derivatives of IP factor can be used as intermediates in synthetic methods for producing IP factor. Said compounds, which are amides, can be obtained by reacting IP factor with a variety of acetylating agents that are known to the person skilled in the art (for instance, March “Advanced Organic Chemistry”, John Wiley & Sons, Inc, 1985). In some cases, these reactions require a protection-deprotection sequence for the alcohol groups. Said acetylating agents may be carboxylic acids, which may require the presence of catalysts such as strong organic acids; acid halides such as acetyl chloride or 9-fluorenylmethyl chloroformate; or acid anhydrides such as acetic anhydride. Preferably said compound is of the formula (I) wherein R₁ is hydrogen and R₂ is 9-fluorenylmethyloxycarbonyl (di-FMOC-IP factor).

The second aspect of the invention is a method for the production of a compound of formula (I) wherein R₁ is hydrogen and R₂ is hydrogen or salts thereof. The method comprises fermentation of a natamycin-producing organism such as Streptomyces natalensis or Streptomyces gilvosporeus. Said compound of formula (I) wherein R₁ is hydrogen and R₂ is hydrogen can be obtained for instance by extraction with a water-immiscible solvent or a partially water-miscible solvent. Preferably said Streptomyces natalensis strain is Streptomyces natalensis ATCC 27448 or an npi mutant capable of producing IP factor (see Examples). Preferably said Streptomyces gilvosporeus strain is Streptomyces gilvosporeus ATCC 13326. Preferably, said solvent is ethyl acetate. However, other solvents such as 1-butanol, tert-butanol, butyl acetate, chloroform, dichloromethane, 1,2-dichloroethane, diisopropyl ether, diethyl ether, isopropyl acetate, methyl acetate, methyl isobutyl ketone, methyl tert-butyl ketone, propyl acetate, toluene, xylene and the like may be used. The person skilled in the art will have no problem selecting the most convenient solvent. Depending on the nature of the solvent, the extraction procedure may be repeated several times, for instance 2 to 10 times, or be performed in continuous countercurrent extraction equipment. The production of said compound of formula (I) wherein R₁ is hydrogen and R₂ is hydrogen generally is from 1 nM to 100 mM, preferably from 10 nM to 10 mM, more preferably from 100 nM to 1 mM.

In one embodiment, the culture broth is concentrated prior to extraction using any of the concentration methods available in the art, such as evaporation, lyophilization or membrane techniques.

In another embodiment, the culture broth, which has been optionally concentrated, is clarified by adding acid to a pH ranging from 1 to 5, preferably 2 to 4, more preferably 2.5 to 3.5. Preferably said acid is hydrochloric acid. However, the person skilled in the art will be aware that other mineral acids (for example sulfuric acid and nitric acid) as well as organic acids (for example acetic acid and formic acid) may be used. Said clarifying process can be performed at temperatures ranging from 0 to 50° C., preferably from 1 to 30° C., more preferably from 2 to 25° C., still more preferably from 3 to 20° C., most preferably from 4 to 10° C.

In another embodiment, solids are removed from the clarified fermentation liquid. Any available solid-liquid separation technique (such as filtration and centrifugation) may be used for this purpose. Prior to extraction, the pH of the clarified liquid is brought to a value ranging between 5 to 9, preferably 6 to 8, more preferably 6.5 to 7.5. Said pH change can be effected with any base known to the person skilled in the art. Examples include ammonium hydroxide, potassium hydroxide, sodium carbonate, sodium hydroxide or other inorganic or organic bases.

In still another embodiment, the IP factor present in the organic phase is further purified using known purification techniques such as carbon treatment, size exclusion chromatography, HPLC-chromatography, hydrophobic interaction chromatography or a combination of two or more of said purification techniques. Preferably, the IP factor-containing organic phase is concentrated using concentration techniques as mentioned above prior to purification.

In an alternative embodiment, the IP factor may be obtained by total synthesis using organic synthetic and/or bio-organic synthetic techniques. One such approach is introduction of the two amino groups in 2,3-bis(hydroxymethyl)-1,4-but-2-enediol, for instance by halogenation, followed by reaction with an azide and followed by reduction of the azide moieties. Yet another approach may be the direct addition of halogen azide to 2,3-bis(hydroxymethyl)-1,4-but-2-enediol, followed by replacement of the remaining halide with an azide and followed by reduction. Said 2,3-bis(hydroxymethyl)-1,4-but-2-enediol may be obtained by allylic hydroxylation of the easily accessible 2,3-dimethyl-2-butene or by reaction of optionally protected dihydroxyacetone with the optionally protected phosphonium salt of 2-bromo-1,3-propanediol.

Other synthetic approaches may be equally suitable and the person skilled in the art will be able to design the most feasible process based on basic chemical knowledge. One particular alternative is reduction of the easily available dihydroxyacetone to give 2,3-bis(hydroxymethyl)-2,3-dihydroxy-1,4-butanediol, the tertiary hydroxyl groups of the latter compound are then converted to amino groups using well-known techniques such as conversion of said tertiary hydroxyl groups into sulfonyl esters followed by replacement with an azide such as a metal azide and subsequent reduction. Optionally, the primary amino groups are protected during these procedures and deprotected afterwards using protecting groups as described in Greene et al. (“Protective Groups in Organic Synthesis”, John Wiley & Sons, Inc, 1991).

A third aspect of the invention is an improved fermentation process for the production of natamycin comprising the addition of a composition comprising an auto inducer to the fermentation medium. Such auto inducers may be any of the butyrolactone class of auto inducers (e.g. the A factor of Streptomyces griseus, the virginia butanolide factors of Streptomyces virginiae and similar compounds isolated from Streptomyces coelicolor, Streptomyces viridochromogenes, Streptomyces bikiniensis, Streptomyces cyaneofuscatus, Vibrio fischeri and other Actinomycetes) or the IP factor or salts thereof. Said fermentation process can be any production process that is based on the use of a Streptomyces strain. Said Streptomyces strain may be any natamycin-producing strain such as Streptomyces natalensis or Streptomyces gilvosporeus. Preferably said natamycin-producing strain is Streptomyces natalensis ATCC 27448 or Streptomyces gilvosporeus ATCC 13326, however other strains are equally suitable. Said Streptomyces strain may also be an amphotericin B-producing strain such as Streptomyces nodosus or a nystatin-producing strain such as Streptomyces noursei. Alternatively, said Streptomyces strain may also be a recombinant strain that is suitable for the production of natamycin or derivatives of natamycin as described in Martin et al. (International Patent Application WO 00/77222). Such derivatives are for instance derivatives that have a double bond instead of an epoxide function between carbon atoms C4 and C5 of the natamycin molecule and/or have an aldehyde, alcohol, or methyl group instead of a carboxyl group at the C12 carbon atom of the natamycin molecule as described in Martin et al. (International Patent Application WO 00/77222).

The person skilled in the art is well aware of the various media that are used for the fermentation of Streptomyces strains; said media are all suitable for the method of the present aspect. Particularly useful media are the complex media such as TSB, MEA, NBG, YED and YEME.

The auto inducer may be added as such. However, the invention also encompasses adding a second organism that produces the auto inducer.

In a preferred embodiment, IP factor or salts thereof may be added in amounts that will lead to an overall IP factor concentration of 5 to 2000 nM, preferably 20 to 1000 nM, more preferably 50 to 400 nM. IP factor or salts thereof can be added during many stages of the fermentation process, for instance in one or multiple batches during the fermentation, before starting the fermentation, after a certain predetermined production level has been reached, admixed to one or more of the components that are added to the fermentation and the like. IP factor or salts thereof may be added as a solid substance and/or dissolved in solvent. Following the completion of the fermentation process, the produced natamycin can be isolated according to any one of the methods known to the person skilled in the art.

A fourth aspect of the invention is an improved fermentation process for the production of natamycin comprising increasing the concentration of an auto inducer by increasing the natural production of said auto inducer by the organism in the fermentation medium.

A fifth aspect of the invention is the use of IP factor in the manufacture of a product by fermentation of a Streptomyces strain. Preferred strains, media and IP factor concentrations and modes of addition are the same as mentioned in the third and fourth aspects of the invention. Preferably the compounds produced by the fermentation process are natamycin, a pigment or extra-cellular enzymes such as cholesterol oxidase.

In a preferred embodiment, IP factor is used to bind to an IP factor-binding protein present in the Streptomyces strain. Said IP factor-binding protein is the so-called repressor-type regulator that represses production of the required product, such as natamycin, in the absence of IP factor. Once IP factor is present, for example through addition as described above, the repressing function of the IP factor-binding protein is blocked and natamycin production is enhanced.

A sixth aspect of the invention is a class of mutants that is defective in natamycin production (npi mutants). Said mutants can be obtained by carrying out mutagenesis techniques on Streptomyces natalensis strains. At least one round of mutagenesis is used. Npi mutants are detected and isolated as a result of their inability to inhibit growth of a fungal test strain. Said test strain can be any strain of fungus whose growth can be inhibited by natamycin. Preferably said test strain is a Candida utilis strain, more preferably said strain is Candida utilis CECT 1061 (see also Example 1).

In one embodiment, said mutagenesis technique is NTG and said Streptomyces natalensis strain is Streptomyces natalensis ATCC 27448.

In another embodiment, the npi mutants are defective in natamycin production but not in IP factor biosynthesis. Said npi mutants restore natamycin production in a mutant that is defective in IP factor biosynthesis. Preferably said npi mutants belong to the classes A, B, C, F or J mentioned in the Examples. More preferably said npi mutants are npi16, npi30, npi71, npi79, npi83, npi85, npi88, npi116, npi140, npi148, npi169, npi178, npi218, npi226, npi235, npi238, npi249, npi275, npi276, npi380 or npi384.

In yet another embodiment, the npi mutants are also defective in IP factor biosynthesis as a result of which they will not produce natamycin unless exogenous IP factor is added. The npi mutants of this embodiment are particularly useful for the determination of IP factor productivity in other strains by growing both strains in complementation tests and observing the absence or presence of natamycin production in said npi mutants that are defective in IP factor biosynthesis. Natamycin production can be detected using a test strain as outlined above. Said test strain can be any strain of which the growth can be inhibited by natamycin. Preferably said test strain is a Candida utilis strain, more preferably said strain is Candida utilis CECT 1061. Preferably said npi mutant is npi287.

### LEGEND TO THE FIGURES

FIG. 1 is the HPLC-analysis of FMOC-derivatized pure IP factor (top panel), A factor (90% purity, middle panel) and A factor (90% purity) mixed with IP factor (bottom panel). Values on the x-axis are in minutes. IP factor FMOC elutes at 11.8 min. Note that the Streptomyces griseus A factor preparation has no traces of IP factor.

FIG. 2 shows natamycin production by Streptomyces natalensis npi287 in response to increasing concentrations of IP factor (100 to 500 nM). Dose-response representation is obtained from measuring points at 0, 110, 166, 220, 333, 446 and 500 nM of IP factor. Y-axis: natamycin-induced inhibition zone diameter in Candida utilis overlays in cm (see Example 5); x-axis: concentration of IP factor in nM.

FIG. 3 shows the stimulation of natamycin production by addition of exogenous IP factor (300 nM) in cultures of the wild type Streptomyces natalensis ATCC 27448, in complex TSB medium. FIG. 3A: Cell dry weight (▪); y-axis: dry weight concentration in mg/ml; x-axis: time in hours. FIG. 3B: (▴) Control without IP factor addition; (O) with IP factor addition; y-axis: natamycin concentration in μg/ml; x-axis: time in hours.

FIG. 4 shows the stimulation of natamycin production by addition of exogenous IP factor (300 nM) in cultures of the wild type Streptomyces natalensis ATCC 27448, in complex NBG medium. FIG. 4A: Cell dry weight (♦); y-axis: dry weight concentration in mg/ml; x-axis: time in hours. FIG. 4B: (▪) Control without IP factor addition; (□) with IP factor addition; y-axis: natamycin concentration in μg/ml; x-axis: time in hours.

FIG. 5 shows the stimulation of natamycin production by addition of exogenous IP factor (300 nM) in cultures of the wild type Streptomyces natalensis ATCC 27448, in complex YEME medium. FIG. 5A: Cell dry weight (▴); y-axis: dry weight concentration in mg/ml; x-axis: time in hours. FIG. 5B: (▪) Control without IP factor addition; (□) with IP factor addition; y-axis: natamycin concentration in μg/ml; x-axis: time in hours.

FIG. 6 shows the time course of formation of IP factor and natamycin in cultures of Streptomyces natalensis ATCC 27448 (wild type) in Streptomyces minimal medium. FIG. 6A: (▴) Dry weight; y-axis: dry weight concentration in mg/ml; x-axis: time in hours. FIG. 6B: (□) IP factor; (▪) natamycin; y-axis: natamycin concentration in μg/ml on the left-hand side and IP factor concentrations in ng/ml on the right-hand side; x-axis: time in hours.

FIG. 7 shows the time course of formation of IP factor and natamycin in cultures of Streptomyces natalensis ATCC 27448 (wild type) in Lechevalier defined medium. FIG. 7A: (▴) Dry weight; y-axis: dry weight concentration in mg/ml; x-axis: time in hours. FIG. 7B: (□) IP factor; (▪) natamycin; y-axis: natamycin concentration in μg/ml on the left-hand side and IP factor concentrations in ng/ml on the right-hand side; x-axis: time in hours.

FIG. 8 shows the time course of formation of IP factor and natamycin in cultures of Streptomyces natalensis ATCC 27448 (wild type) in complex TSB medium. FIG. 8A: (▴) Dry weight; y-axis: dry weight concentration in mg/ml; x-axis: time in hours. FIG. 8B: (□) IP factor; (▪) natamycin; y-axis: natamycin concentration in μg/ml on the left-hand side and IP factor concentrations in ng/ml on the right-hand side; x-axis: time in hours.

FIG. 9 is the same as FIG. 8 with the exception that instead of TSB medium, NBG medium is used.

FIG. 10 is the same as FIG. 8 with the exception that instead of TSB medium, YEME medium is used.

FIG. 11 is the same as FIG. 8 with the exception that instead of TSB medium, YED medium is used.

FIG. 12 is the HPLC analysis showing the absence of IP factor in complex NBG medium at inoculation time (top panel); NBG medium supplemented with 0.2 □g/ml IP factor (middle panel) and NBG culture broth after 48 h inoculated with Streptomyces natalensis ATCC (bottom panel). The IP factor-FMOC peak is shaded. An inclined arrow indicates the expected position of IP factor-FMOC in the top panel. Similar results were obtained in TSB, YEME and YED medium.

### EXAMPLES

In the examples given below, the following strains were used: The wild type Streptomyces natalensis ATCC 27448 was used as the parental strain to isolate different npi mutants. Streptomyces natalensis cultures were maintained in solid TBO sporulation medium (containing 20 g tomato paste per liter; 25 g oat meal per liter; and 25 g agar per liter) as described by Aparicio et al. (Chem. Biol. 7, 895-905, 2000). Candida utilis CECT 1061 (synonym Pichia jadinii) was used as test strain in the bioassay of the antifungal activity of natamycin. Cultures of Streptomyces griseus IFO 13350 (formerly described as Streptomyces bikiniensis IFO 13350) were used to produce A factor in YMPG medium (Horinouchi et al., J. Antibiot. 38, 636-641, 1985). Streptomyces griseus HH1 (an A factor negative strain) was used to quantify the A factor inducing activity.

### Example 1

### Mutagenesis and Isolation of Non-Producing Clones

Spores of Streptomyces natalensis ATCC 27448 (about 106 spores/ml) were suspended in 0.05 M Tris-maleate buffer, pH 9.0 and mutated with N-methyl-N′-nitrosoguanidine (1 mg/ml) for 20 min at 30° C. Under those conditions the mortality after 20 min was about 50%.

The mutated spores were washed, diluted and plated in YED medium and incubated at 28° C. When the colonies started to grow (after 24 h) agar plugs (7 mm diameter) containing individual colonies were cut out from the plates, incubated under high aw (humidity) conditions for an additional 24 h and the natamycin production by each clone was assayed on a lawn of Candida utilis. Mutants that did not produce inhibition zones, i.e. mutants wherein the visible ring of inhibition surrounding the agar plug was smaller than 1 mm, were selected. The lack of production of natamycin of the selected mutants was confirmed in liquid cultures in SPG medium (Gil et al., J. Gen. Microbiol. 131, 1279-1287, 1985). Mutants that did not revert in liquid medium cultures were further analyzed by complementation tests.

A total of 384 non-producer (npi) mutants impaired in natamycin biosynthesis (npi1 to npi384) were isolated in a first round of selection after NTG mutagenesis as described above. Some of them reverted or were unstable. After several rounds of selection (see Example 3) 35 stable npi mutants were selected (Table 1) and assayed in pair-wise complementation tests on solid YED medium. For the complementation of npi mutants of Streptomyces natalensis a co-synthesis method was used (see Example 3). Two npi (non-producer) mutants A and B were unable to produce natamycin when assayed separately as agar-plug cultures on a lawn of Candida utilis but one of them (A, the converter) regains natamycin production when it was grown close together to the “donor” strain-B.

Based on the results of the complementation tests, the mutants were divided into 11 classes (A to K in Table 1). A group of seven mutants (npi12, npi54, npi64, npi86, npi98, npi6 and npi137) (class H in Table 1) were unable to complement any other mutant class or vice versa.

Non-producer mutants of classes A, B, C, F, and J were all able to complement npi287 (class G, Table 1). The results of these complementation tests indicated that npi mutants of classes A, B, C, F and J were able to produce an inducer substance that complements natamycin production in npi287 and that these mutants were blocked later in the biosynthetic pathway. Mutant npi287 responded clearly to the spent culture broth of the wild type Streptomyces natalensis ATCC27448 and was therefore used as tester (converter) strain for the presence of the IP factor. Mutant classes D, E, K and I failed to complement the inducer requiring class G and therefore may also contain mutations related to the IP factor biosynthesis.

TABLE 1
Complementation classes of npi (non-producer
mutants) of Streptomyces natalensis

| | | Complemented |
|---|---|---|
| Classes | | classes |
| A | npi380 | B, C, D, E, G, J, K |
| B | npi16, npi235 | A, C, E, G |
| C | npi275 | A, B, D, G, I, J |
| D | npi31 | A, C |
| E | npi38 | A, B |
| F | npi30, npi71, npi83, npi85, npi116, | G |
| | npi140, npi148, npi178, npi226, | |
| | npi238, npi249, npi276 | |
| G | npi287 | A, B, C, F, J |
| H | npi6, npi12, npi54, npi64, npi86, npi98, npi137 | None |
| I | npi22, npi255 | C |
| J | npi79, npi88, npi169, npi218, npi384 | A, C, G |
| K | npi39, npi271 | A |

### Example 2

### Culture Media and Quantification of Natamycin Production in Liquid Cultures

Four different complex media were used for quantification of the IP factor concentration and its relationship to natamycin production. These include: NBG medium (OXOID) supplemented with glucose (5 g/l); YEME medium (yeast extract 3 g/l; peptone 5 μl; malt extract 3 g/l and glucose 10 g/l); TSB medium (DIFCO) and YED medium (yeast extract 10 g/l; glucose 10 g/l). In addition two defined media were also used to quantify the inducer production: Streptomyces MM (Kieser et al. in “Practical Streptomyces Genetics”, John Innes Foundation, Norwich, UK, 2000) and Lechevalier defined medium (Martin and McDaniel, Eur. J. Appl. Microbiol. 3, 135-144, 1976).

The production of natamycin in liquid cultures was routinely quantified by spectrophotometric determination at 319 nm. A 0.5 ml aliquot of the culture was extracted with 5 ml of methanol and diluted with distilled water; the concentration of natamycin was quantified as described previously (Aparicio et al. (Chem. Biol. 7, 895-905, 2000)) using a pure sample of natamycin (Sigma Chem. Co) as standard.

### Example 3

### Complementation Tests

Complementation tests were performed between pairs of 31 stable non-producer mutants using standard co-synthesis methods in solid YED medium. Each pair of npi mutants was grown as lawn cultures. Agar plugs were taken out from each of the growth zones and the production of natamycin was bioassayed using Candida utilis as the sensitive organism. Positive complementation was clearly detected by the production of natamycin when the two non-producer mutants were placed close to each other, whereas control plugs from each of the two non-producer mutants gave no inhibition zone, when assayed separately.

### Example 4

### Extraction and HPLC Purification of the IP Factor to Homogeneity

The culture broth (15 liters) from Streptomyces natalensis wild type strain grown for 24 h in YED medium in a Braun Biostat C fermentor was concentrated 10-fold in a vacuum evaporator. The concentrated broth was clarified by precipitating the proteins with 6M HCl at pH 3.0 in the cold room. The clarified broth was adjusted to pH 7.0 with concentrated NaOH and extracted. Surprisingly however, a significant proportion of IP factor remained in the aqueous phase after ethyl acetate extraction at either acidic, neutral or slightly basic (pH 7.5) pH values. On the contrary to butyrolactones, in repeated attempts to extract the inducer with chloroform a significant part of the IP factor (about 80%) was found to remain always in the aqueous phase, suggesting that it was a rather hydrophilic molecule. Enough IP factor could be extracted by repeated extractions with ethyl acetate to allow a good purification. The organic phases were then collected and concentrated to dryness under vacuum and the resultant product was dissolved in 100 ml of 10% methanol (v/v) and applied to an active carbon column (30×3 cm) previously equilibrated with the same solvent (10% methanol, v/v). The retained compounds including the IP factor were fractionated by stepwise elution (flow 2 ml/min) with 50% methanol (v/v), 100% methanol, 10% ethyl acetate in methanol (v/v), 50% ethyl acetate in methanol (v/v), and pure ethyl acetate (100%). Bioassays of the IP factor showed that it eluted in the second fraction (100% methanol). The IP factor-containing fraction was then concentrated, applied onto a Sephadex GIO column (2000×1 cm) and eluted with distilled water (flow 0.8 ml/min). This size exclusion chromatography yielded 40 ml of active fractions.

After elution from the Sephadex G10 column the biologically active fractions were purified by reverse phase HPLC chromatography using a Waters 600 unit coupled to a PDA 996 detector equipped with a Polarity C18 column (3.9×150 mm; particle size, 5 mm). The IP factor elutes at a retention time of 2.5 min with a mobile phase mixture consisting of a linear gradient of acetonitrile-water (from 1:99, v/v at time 0 to 70:30, v/v at 15 min). The pure IP factor was derivatized with FMOC (9-fluorenylmethyl chloroformate) as described by Sim and Perry (Glycoconjugate J. 14, 661-668, 1997).

As a control A factor was purified from cultures of Streptomyces griseus IFO 13350. As expected, A factor was clearly purified using this protocol and eluted in the 100% methanol fraction.

### Example 5

### Determination of IP Factor Biological Activity

Mutant npi287 Recovers Natamycin Production when Supplemented with Streptomyces natalensis Wild Type Culture Broth or with A Factor from Streptomyces griseus

Since initial studies indicated that mutant npi287 recovered natamycin production in co-synthesis experiments with different Streptomyces natalensis mutant strains, its complementation with spent culture broth of the parental strain Streptomyces natalensis ATCC 27448 was tested. Results showed that mutant npi287 recovered full natamycin production levels when supplemented with culture broths of the Streptomyces natalensis wild type strain grown for 24 h in either YED, NB or YEME media suggesting that the IP factor was secreted by the wild type strain. In a bioassay it was shown that IP factor has a strong inducing activity on Streptomyces natalensis npi287 whereas its acetylated derivative has no inducing effect. The Streptomyces griseus A factor has also some inducing activity on Streptomyces natalensis npi287.

The Purified IP Factor is Different from A Factor

The Streptomyces griseus 35570 A factor was purified by following its biological activity on the test strain Streptomyces griseus HH1, a mutant lacking streptomycin production due to its deficiency in A factor biosynthesis. The purified Streptomyces griseus A factor containing fraction elicited natamycin production in the Streptomyces natalensis npi287 mutant as shown above, as also did the crude culture broth of streptomycin-producing parental strain of Streptomyces griseus 35570. These results indicated that natamycin production by the npi287 strain responds to A factor.

Interestingly, the opposite tests were negative. Neither the wild type Streptomyces natalensis culture broth nor the HPLC-pure IP factor restored streptomycin production to the Streptomyces griseus HH1 mutant, further indicating that the IP factor is different from A factor and specific for Streptomyces natalensis.

The pure IP factor did not stimulate sporulation of the wild type Streptomyces natalensis or the npi287 mutant, at difference of the well-known stimulation of sporulation of Streptomyces griseus HH1 exerted by A factor.

These results, together with the susceptibility of the IP factor (but not of A factor) to be derivatized with FMOC, clearly supported the proposal that the nature of both compounds is different.

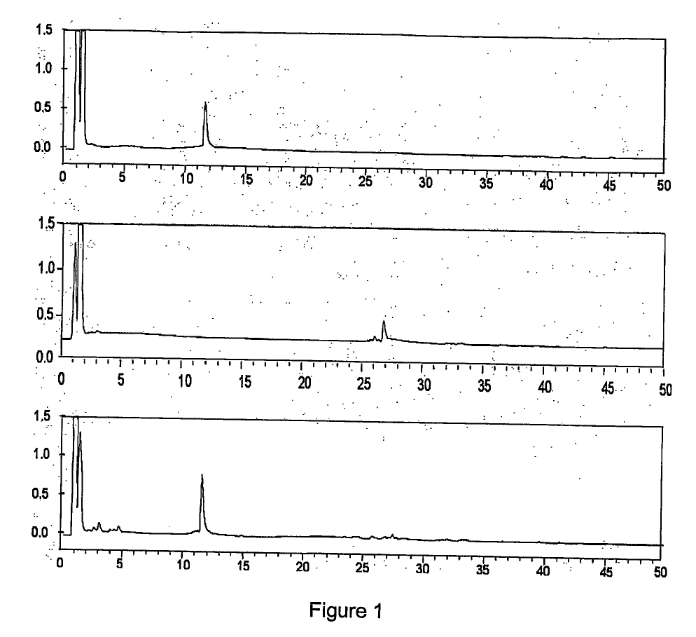

To study whether any traces of IP factor might be produced by the Streptomyces griseus strain, comparative HPLC analysis of the pure IP- and A factor containing fractions were performed. Results (FIG. 1) showed that the active IP factor derivatized with FMOC eluted at 11.0 min (FIG. 1, top and bottom panels), whereas no peak could be observed in the A factor chromatogram after FMOC derivatization (FIG. 1, middle panel). The ability of the IP factor to react with FMOC suggested the presence of amino groups in its structure, while the lack of reaction of A factor under the same conditions confirmed the absence of such groups in the A factor structure.

Dose-Response: the IP Factor Works at Low Concentrations

The biological activity of IP factor was determined by its ability to induce natamycin production by mutant strain Streptomyces natalensis npi287 in solid SPG medium. After allowing growth of Streptomyces natalensis npi287 for 2 days at 30° C., samples of culture broths (100 ml) or different fractions from IP factor purification were added to wells (7 mm diameter) in the agar layer. The plate was overlaid with a culture of Candida utilis and incubated for 24 h at 28° C. The increment of the diameter of the natamycin inhibition zone after induction of natamycin in strain npi287 was proportional to the amount of IP factor in the sample.

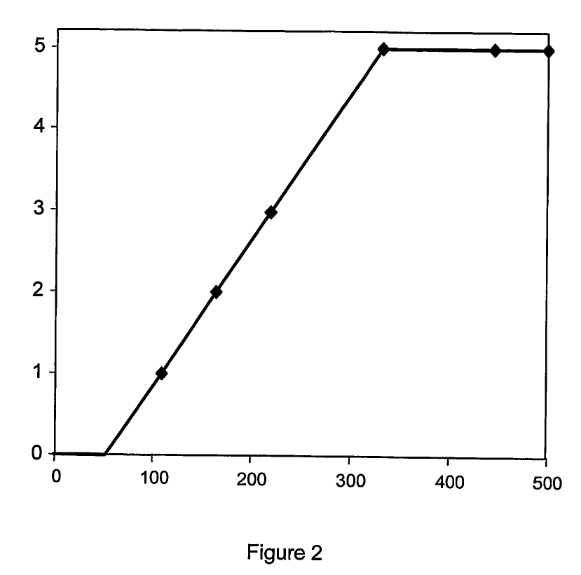

The availability of the pure IP factor made it possible to quantify its inducing effect using the standard inducer assay with the npi287 strain (FIG. 2). The npi287 strain clearly responded to IP factor concentrations of 100 nM and the antibiotic production showed a linear response up to 350 nM. The IP factor increased two-fold the diameter of natamycin inhibition zone (to above 50 millimeters) at concentration of 350 nM and the assay was saturated at concentrations above 400 nM. A threshold level of IP factor of about 50 nM was always required to detect the induction of natamycin production. These results are consistent with a cooperative effect typical of a “quorum-sensing” type molecule.

The Production of Natamycin by the Wild-Type Strain Streptomyces natalensis ATCC 27448 is Stimulated by Exogenous IP Factor

In quorum sensing systems the autoinducer signal is secreted by some cells in the culture population and the inducer is incorporated by other cells to trigger differentiation or other biochemical switches.

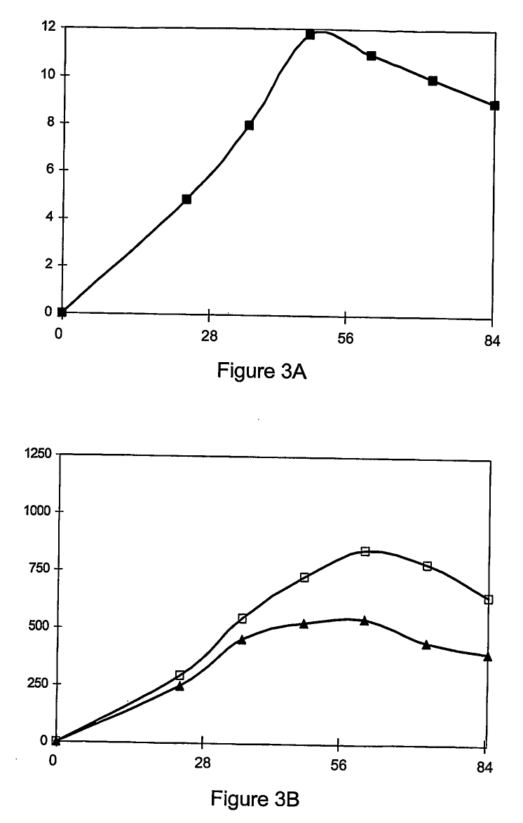

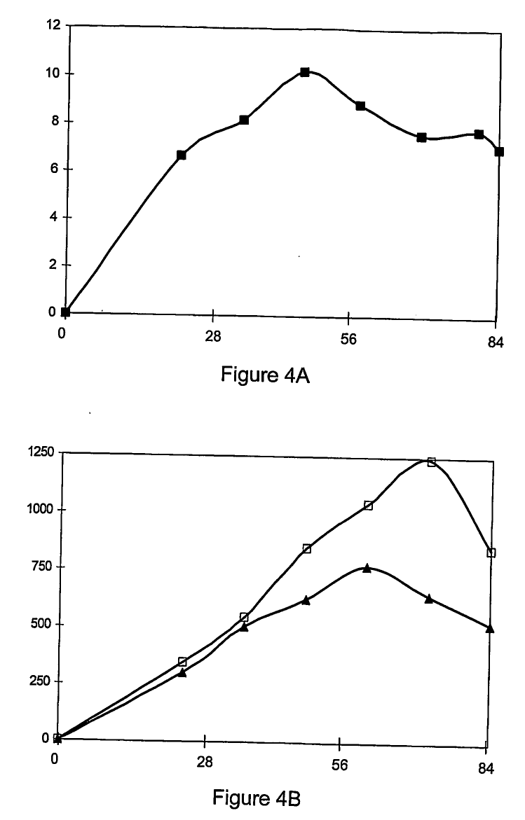

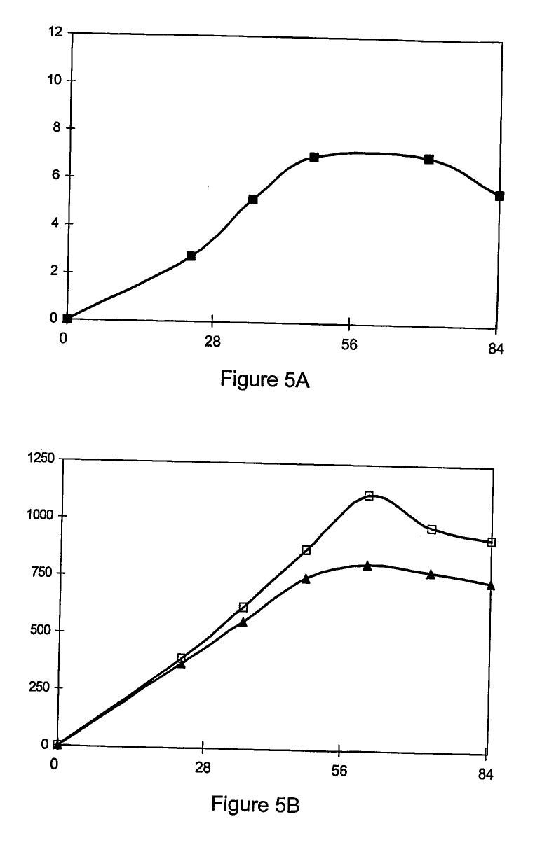

As shown in FIGS. 3, 4 and 5, the production of natamycin by the parental strain Streptomyces natalensis ATCC 27448 in NBG, TSB and YEME media was stimulated by the addition of 300 nM IP factor in the three culture media. Since the wild type synthesizes endogenous IP factor, the stimulatory effect of exogenous inducer indicates that it is limiting for natamycin biosynthesis in the wild type. Furthermore the results indicate that IP factor is taken up by Streptomyces natalensis or at least it triggers a signal cascade leading to overproduction of natamycin.

### Example 6

### Kinetics of Production of IP Factor in Cultures of Streptomyces natalensis in Defined and Complex Media

The time of synthesis of the IP factor is relevant to trigger the onset of natamycin biosynthesis. Furthermore, the level of IP factor formed may be limiting for total natamycin accumulation. To analyze the time-course of IP factor biosynthesis, Streptomyces natalensis ATCC 27448 was grown in two defined media, namely MM for Streptomyces (Kieser et al. in “Practical Streptomyces Genetics”, John Innes Foundation, Norwich, UK, 2000) and Lechevalier (Martin and McDaniel, Eur. J. Appl. Microbiol. 3, 135-144, 1976), and in four complex media TSB, MEA, NBG, YED and YEME that are known to support high natamycin production.

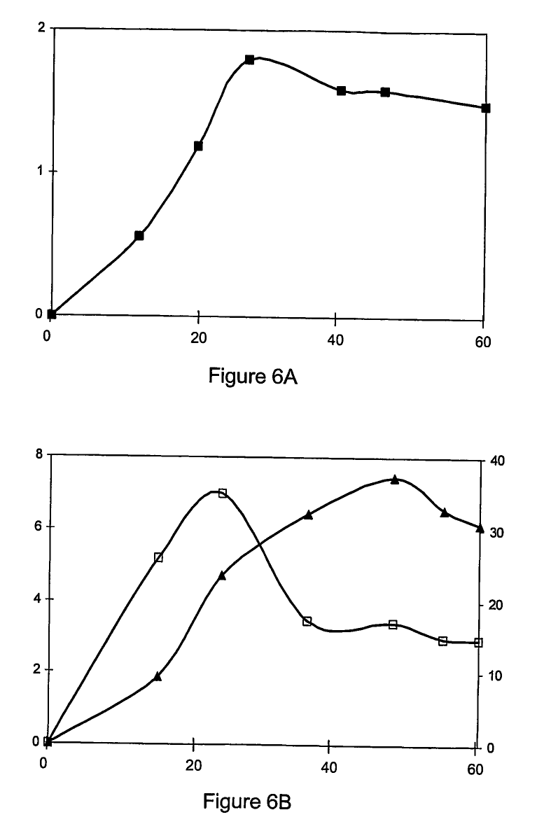

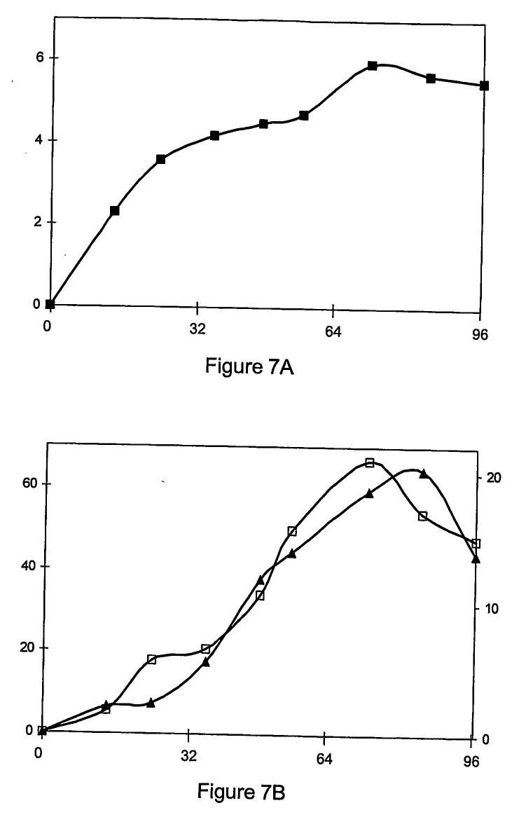

The two defined medium supported low yields of natamycin (FIGS. 6 and 7). In both Streptomyces MM and Lechevalier MM, IP factor was synthesized in parallel to growth and a peak (20 to 40 ng/ml) was observed at the end of rapid growth phase (equivalent to the exponential growth phase of unicellular bacteria). Biosynthesis of natamycin in these two defined media was parallel to the growth of the culture with a delay of about 12 h. These results indicate that the formation of IP factor is related to the growth phase and it precedes natamycin production.

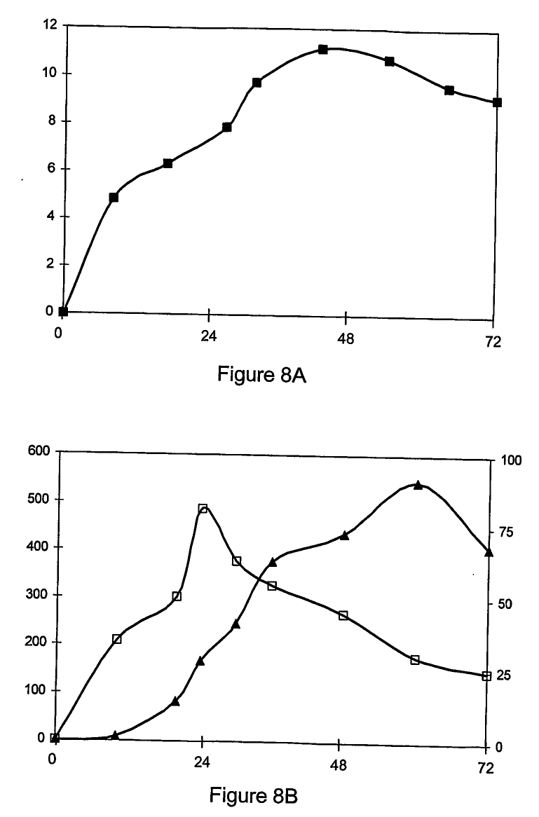

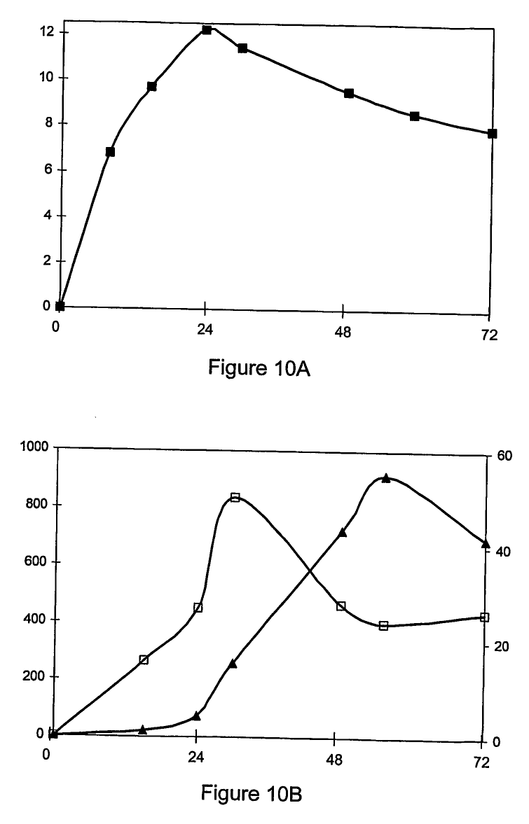

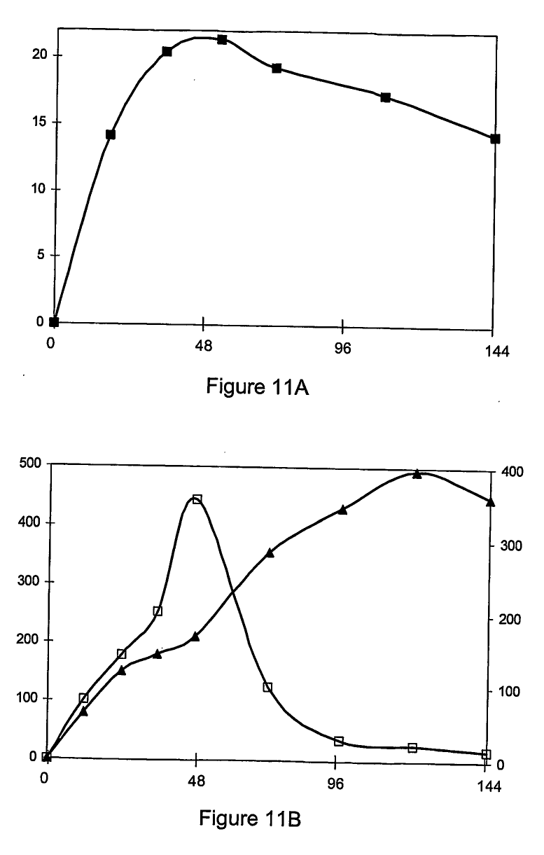

All complex media were found to support much higher levels of natamycin production (FIGS. 8 to 11) ranging from 550 mg/ml in TSB medium (FIG. 8B) to 850 mg/ml in YEME medium (FIG. 10B). In the four complex media the IP factor was formed much earlier than natamycin coinciding with the growth phase and reached levels of IP factor between 50 ng/ml in YEME medium and 80 ng per ml in TSB medium that were clearly higher (about two- to three-fold) than the level of IP factor in defined media. A special mention deserves the YED medium in which IP factor accumulated to levels of 450 ng/ml. Production of natamycin in this medium was also high (500 mg/ml) but did not match the large increase observed in IP factor. These results indicate that biosynthetic steps other than the level of IP factor are limiting for natamycin production above IP factor saturation levels in this medium.

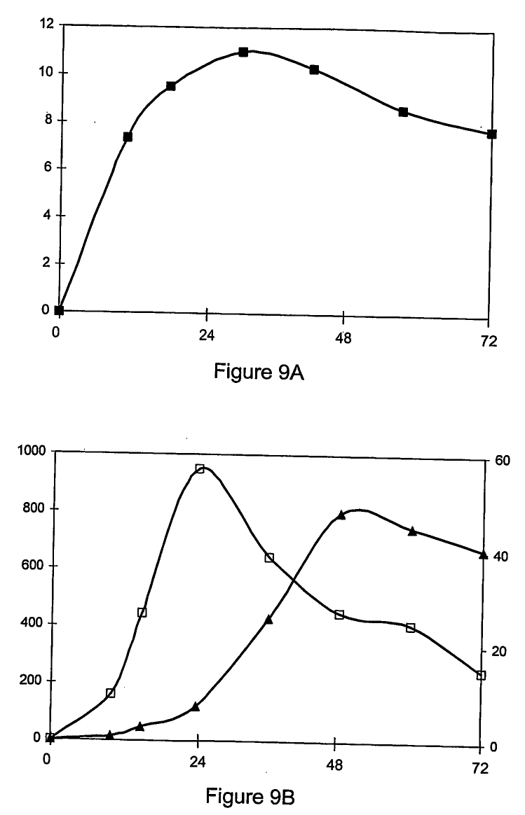

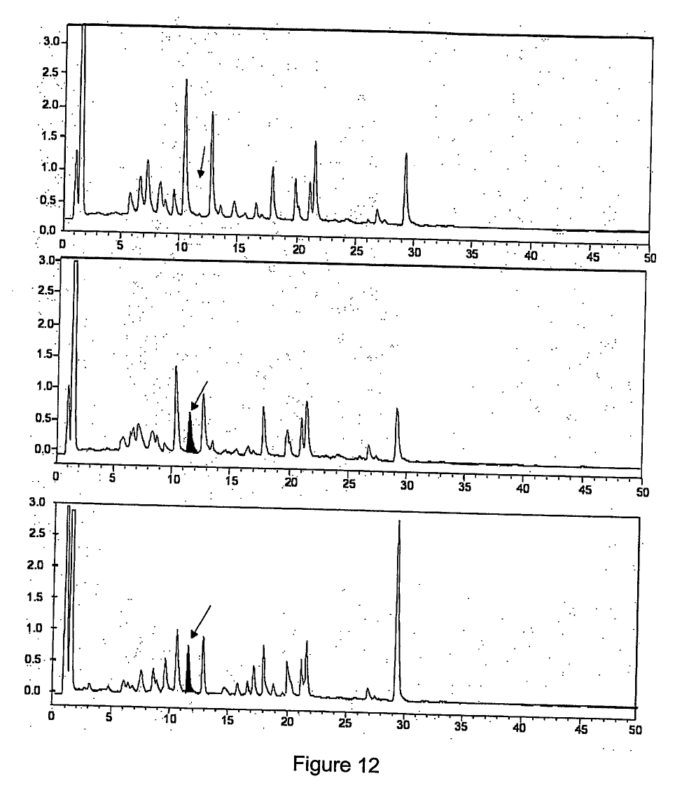

To confirm that the IP factor is produced during the culture of Streptomyces natalensis and is not present in the complex media used in these experiments all media were tested by HPLC before inoculation and after 48 h of incubation with Streptomyces natalensis (results in FIG. 12). There was no IP factor in the complex medium before inoculation and it clearly accumulated after 48 h of incubation in this medium. To confirm the absence of IP factor the NBG medium was primed with pure IP factor. As shown in FIG. 9, middle panel, the HPLC elution profile of the IP factor supplemented culture medium confirmed the lack of this molecule in the starting culture medium.

### Example 7

### NMR Spectroscopy and Mass Spectrum Analysis

The structure elucidation of IP factor was established by NMR spectroscopy, using a combination of 1 D NMR methods (¹H-NMR and ¹³C-NMR) and 2D shift-correlated NMR techniques (HMQC-HSQC and HMBC) for the complete ¹H and ¹³C signal assignments. NMR spectra were recorded in D₂O at room temperature using a Bruker WM 500 spectrometer [500 MHz (¹H NMR) and 125 MHz (¹³C NMR)]. Chemical shifts are given on the 6-scale and were referenced to the solvent and to dioxane as internal signal. The pulse programs of the 2D experiments were taken from the Bruker software library and the parameters were as follows: 500/125 MHz gradient-selected HMQC spectra, relaxation delay D₁=1.5 s; evolution delay D₂=3.33 ms; delay for evolution of long range coupling (HMBC) D₆=60 ms. The ES+ mass spectrum was recorded on a HP 1100-MSD using CF₃COOH 0.1% as the source of ionization.

The analysis of the ¹H NMR spectrum of IP factor showed only a signal at δ 3.71 ppm as a singlet whereas its ¹³C NMR spectrum contained two signals, which were assigned to one methylene group (CH₂, δ58.76 ppm) and one quaternary carbon atom at δ60.87 ppm. This was confirmed by means of a DEPT experiment. The assignment of the carbon atom of the methylene group was carried out by using a HMQC spectrum, which showed a correlation peak via ¹J_{H,C} with the methylene protons at δ 3.71 ppm. In the HMBC spectrum one key correlation peak via ³J_{C,H} was obtained between the methylene protons and the quaternary carbon atom at δ 60.87 ppm.

TABLE 2
¹H NMR and ¹³C NMR data of IP factor
(in D₂O; chemical shifts in δ ppm)

| | |
|---|---|
| pH = 5.20 | pH = 8.05 |

| Group | ¹H | ¹³C | ¹H | ¹³C |
|---|---|---|---|---|
| CH₂ | 3.71(s) | 58.76 | 3.66(s) | 59.44 |
| C | | 60.87 | | 59.93 |

The low-field nature of the chemical shifts (δ_{H} and δ_{C}) of the methylene group suggests the presence of an oxygenated group (—CH₂OH). This was confirmed by comparison with the NMR spectra of the acetylated derivative of IP factor (IPa). The ¹H NMR spectrum of the acetylated derivative (recorded in CDCl₃) showed two singlets at δ 1.25 and 4.43 ppm, which were attributed to a methyl group (CH₃COO—) and a methylene group (CH₃COOCH₂—), respectively; and the ¹³CNMR spectrum showed four signals at δ 20.70 ppm (CH₃), δ 58.10 ppm (C), δ 62.71 ppm (CH₂) and δ 170.60 ppm (CO).

The downfield shift of the quaternary carbon atom and the variation of the chemical shifts by the pH change (see Table 2), suggested the presence of an amine group. This was confirmed by means of the mass spectrum. The IP factor gave an ion at m/z 91 [M+2H]⁺/2 on the positive electro spray (ES+) indicating a double-charged species. On the basis of all available data the structure of the IP factor is proposed to be 2,3-diamino-2,3-bis(hydroxymethyl)-1,4-butanediol.

1. A compound of the formula (I): 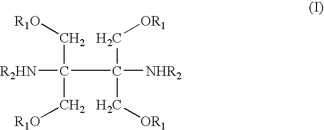
wherein:
each R₁ is hydrogen, optionally substituted alkyl, substituted silyl or —C(O)(R₃) wherein R₃ is hydrogen, optionally substituted alkyl, or optionally substituted aryl; and
each R₂ is hydrogen, optionally substituted alkyl, substituted silyl or —C(O)(R₃) wherein R₃ is hydrogen, optionally substituted alkyl, optionally substituted aryl or OR₄ wherein R₄ is optionally substituted alkyl, or optionally substituted aryl; or a salt thereof.

2. A compound according to claim 1, wherein R₁ is —C(O)(R₃) and wherein R₃ is methyl, ethyl, n-propyl or isopropyl.

3. A compound according to claim 1, wherein R₂ is —C(O)(R₃) and wherein R₃ is methyl, ethyl, n-propyl or isopropyl.

4. A compound according to claim 1, wherein R₂ is —C(O)(OR₄) and wherein R₄ is methyl, ethyl, n-propyl or isopropyl.

5. A compound according to claim 1, wherein R, is hydrogen and R₂ is hydrogen.

6. A process for the production of a compound according to claim 5 comprising fermenting a Streptomyces strain capable of producing said compound.

7. A process according to claim 6, further comprising isolating said compound from the mixture obtained and/or purifying the isolated compound.

8. A process according to claim 6 or 7, wherein the Streptomyces strain is Streptomyces natalensis or a mutant of Streptomyces natalensis that is impaired in natamycin biosynthesis but not impaired in biosynthesis of said compound.

9. A fermentation process for the production of natamycin by a Streptomyces strain comprising adding a composition comprising an auto inducer to the fermentation medium.

10. A fermentation process for the production of natamycin by a Streptomyces strain comprising increasing the concentration of an auto inducer in the fermentation medium by increasing the natural production of said auto inducer by said Streptomyces strain.

11. A process according to claim 9, wherein the auto inducer is a butyrolactone auto inducer.

12. A process according to claim 9, wherein the auto inducer is a compound of the formula (II):

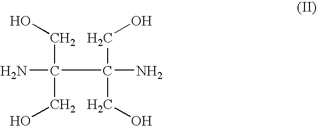

13. A process according to claim 11, wherein the auto inducer is 2-(6′-methyl-heptanoyl)-3R-hydroxymethyl-4-butanolide.

14. Use of a compound according to claim 1 in the manufacture of a product by fermentation of a Streptomyces strain.

15. A use according to claim 14, wherein said compound binds to an IP factor-binding protein present in said Streptomyces strain.

16. A use according to claim 14, wherein said Streptomyces strain is Streptomyces natalensis.

17. A use according to claim 14, wherein said product is a secondary metabolite, a pigment, a protein or a peptide.

18. A use according to claim 17, wherein said product is natamycin.

19. A use according to claim 14, wherein said compound is added to the fermentation medium, the production of said compound by said Streptomyces strain is increased and/or a second strain that produces said compound is added to the fermentation medium.

20. A use of according to claim 14, wherein the overall concentration of the compound in the fermentation medium is from 50 to 400 nM.

21. A Streptomyces strain that is defective in natamycin production and that is capable of producing a compound according to claim 5.

22. A Streptomyces strain that is defective in production of a compound according to claim 5 and that is capable of producing natamycin in the presence of said compound.
